(19) United States
(12) Patent Application Publication
(10) Pub. No.: US 2008/0027420 A1
(43) Pub. Date: Jan. 31, 2008

(54) Cryosurgical Imaging and Monitoring Systems

(75) Inventors: Ling Wang, Maple Grove, MN (US); Jia Hua Xiao, Maple Grove, MN (US); Douglas A. Devens, Highland Park, IL (US); David W. Vancelette, San Diego, CA (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 11/828,095

(22) Filed: Jul. 25, 2007

Related U.S. Application Data
(60) Provisional application No. 60/820,288, filed on Jul. 25, 2006.

(51) Int. Cl. A61B 18/02

(52) U.S. Cl. 606/21

(57) ABSTRACT

Improved imaging and monitoring systems for use with a closed loop cryosurgical system. As described herein, various systems can be used alone or in conjunction with one another to plan and/or monitor cryosurgical procedures in order to improve cryosurgical outcomes. These systems can include computer assisted planning systems, non-ultrasound based imaging systems and temperature monitoring systems utilized individually or in combination. Through the use of these systems, the precision by which cryosurgical procedures are performed are enhanced.

### PRIORITY CLAIM

The present application claims priority to U.S. Provisional Application No. 60/820,288, filed Jul. 25, 2006 and entitled, “CRYOSURGICAL IMAGING AND MONITORING SYSTEMS, which is herein incorporated by reference in its entirety.

### FIELD OF THE INVENTION

The present disclosure relates to cryosurgical systems for use in the treatment of cancerous tumors or lesions, and more particularly to imaging and monitoring systems for use in cryosurgical systems.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer and benign prostate disease. Cryosurgery also has gynecological applications. In addition, cryosurgery may be used for the treatment of a number of other diseases and conditions including breast cancer, liver cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

Cryosurgery is typically carried out under ultrasound guidance to monitor the size and positioning of ice balls with respect to targeted tissue. Ultrasound can be used to visualize the process of freezing during cryosurgery as the interface between frozen tissue and non-frozen tissue is associated with a change in acoustic impedance that reflects ultrasound waves, allowing the interface to be depicted. However, ultrasound can be difficult to use and currently is regulated for use only by radiologists who have received specialized training with it. In addition, ultrasound can create a “shadow region” that obscures vision behind the ice ball. Thus if growth of the ice ball is non-uniform, the lack of vision within the shadow region can lead to vital organs being accidentally frozen.

### SUMMARY OF THE INVENTION

The present disclosure is directed to improved imaging and monitoring systems for use with a closed loop cryosurgical system. As described herein, various systems can be used alone or in conjunction with one another to plan and/or monitor cryosurgical procedures in order to improve cryosurgical outcomes. These systems can include computer assisted planning systems, non-ultrasound based imaging systems and temperature monitoring systems utilized individually or in combination. Through the use of these systems, the precision by which cryosurgical procedures are performed is enhanced.

In one aspect of the present disclosure, a cryosurgical system can utilize a computer assisted planning system and the related methods of implementing the computer assisted planning system to promote cryosurgical treatment uniformity within captured, targeted tissue. The computer assisting planning system can use finite element simulation to tessellate the surface of a captured image of a region to be cryosurgically treated with uniformly sized hexagons. Once these hexagons have been simulated, the center of each hexagon can be used as a placement recommendation or target for a cryoprobe. A guide or template can be used to align the cryoprobes with the hexagons so as to guide them to the placement recommendations.

In another aspect of the present disclosure, a cryosurgical system can use a non-ultrasound based imaging system to visualize a cryosurgical treatment region in real-time. Representative non-ultrasound based imaging systems can utilize either electrical impedance tomography (“EIT”) or near-infrared imaging (“N-IR”). EIT measures electrical resistance across gaps between electrodes placed on the body or needles placed in the body. N-IR measures near infrared absorbance between light fibers placed inside or outside the body. Using the measurements obtained with either EIT or N-IR imaging, a computer program can be used with either system to calculate and depict ice ball location and size at any point in time based on these measurements.

In yet another aspect of the present disclosure, a cryosurgical system can be monitored and controlled utilizing a temperature monitoring system and associated algorithms. The temperature monitoring system can use cryoprobes having servo-actuated valves to control the temperature at the cryoprobe tips as well as thermocouples inserted into desired areas within the prostate. The temperature monitoring system can use a proportional-integral-derivative control to monitor the behavior of the system. The servo-actuated valves can then be rapidly adjusted in order to maintain the desired temperature.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a cryosurgical system according to the present disclosure.

FIG. 2 is a flow chart illustrating a representative computer aided planning procedure for use with a cryosurgical system according to the present disclosure.

FIG. 3 is a flow chart illustrating a representative cryosurgical treatment procedure according to the present disclosure.

FIG. 4 is a flow chart illustrating a representative temperature monitoring algorithm according to the present disclosure.

### DETAILED DESCRIPTION OF THE PREFERRED EMBODIMENTS

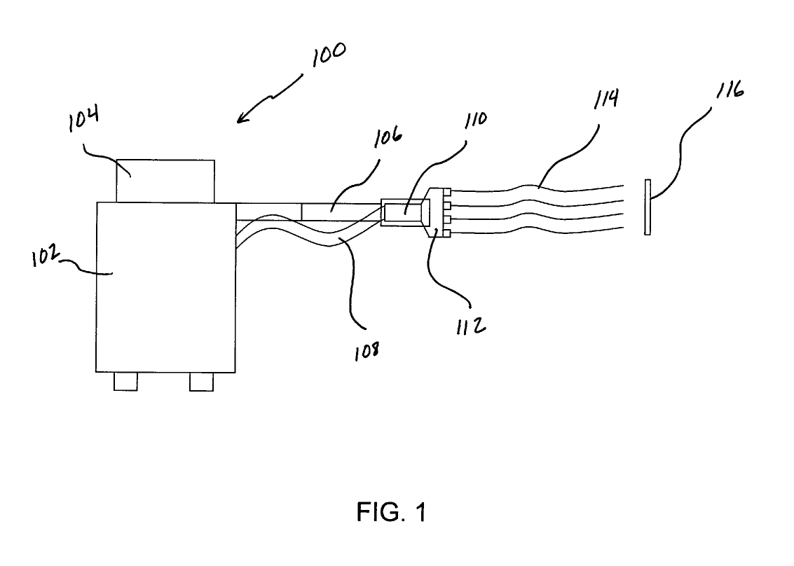

A closed loop cryosurgical system 100 according to the present disclosure is illustrated in FIG. 1. Cryosurgical system 100 can include a refrigeration and control console 102 with an attached display 104. Control console 102 can contain a primary compressor to provide a primary pressurized, mixed gas refrigerant to the system and a secondary compressor to provide a secondary pressurized, mixed gas refrigerant to the system. The use of mixed gas refrigerants is generally known in the art to provide a dramatic increase in cooling performance over the use of a single gas refrigerant. Control console 102 can also include controls that allow for the activation, deactivation, and modification of various system parameters, such as, for example, gas flow rates, pressures, and temperatures of the mixed gas refrigerants. Display 104 can provide the operator the ability to monitor, and in some embodiments, adjust the system to ensure it is performing properly and can provide real-time display as well as recording and historical displays of system parameters. One exemplary console that can be used with an embodiment of the present invention is used as part of the Her Option® Office Cryoablation Therapy available from American Medical Systems of Minnetonka, Minn.

With reference to FIG. 1, the high pressure primary refrigerant is transferred from control console 102 to a cryostat heat exchanger module 110 through a flexible line 108. The cryostat heat exchanger module 110 can include a manifold portion 112 that transfers the refrigerant into and receives refrigerant out of one or more cryoprobes 114. The cryostat heat exchanger module 110 and cryoprobes 114 can also be connected to the control console 102 by way of an articulating arm 106, which may be manually or automatically used to position the cryostat heat exchanger module 110 and cryoprobes 114. Although depicted as having the flexible line 108 as a separate component from the articulating arm 106, cryosurgical system 100 may incorporate the flexible line 108 within the articulating arm 106. A positioning grid 116 can be used to properly align and position the cryoprobes 114 for patient insertion.

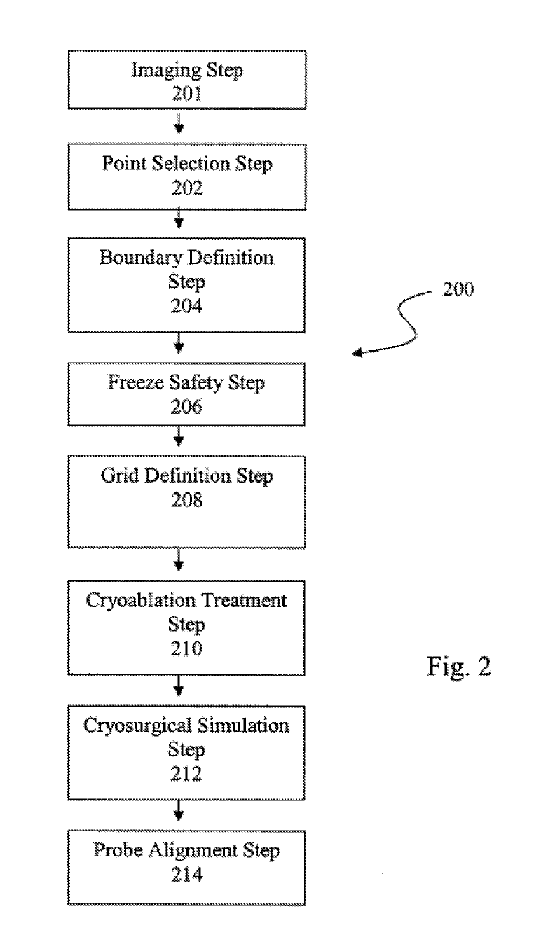

A cryosurgical system according to the present disclosure can utilize a computer-assisted planning procedure 200 illustrated graphically in FIG. 2. The computer-assisted planning procedure 200 can be used to plan and predict a cryosurgical procedure prior to treatment. A cross-sectional image of a region to be treated such as, for example, the cross-section of the prostate or other target tissue is first captured at an imaging step 201 using an imaging technique such as, for example, trans-rectal ultrasound (TRUS), computed tomography (CT), or magnetic resonance imaging (MRI), or other suitable imaging technique. At a point selection step 202, the user can select a number of points, for example eight, from the captured image which a software portion of the computer-assisted planning system uses at boundary definition step 204 to interpolate a freeze boundary around the cross-section image. In a prostate application, a circle must also be placed over the urethra at a freeze safety step 206 to ensure it is not in the freeze zone defined by the freeze boundary. The software portion can include a finite element simulation algorithm to tessellate the surface of the generated prostate shape with uniformly sized hexagons at a grid definition step 208. The size of the hexagons should have the same chord diameter as an iceball generated by a cryoprobe would have after a set period of time. This period of time can be specified by the user or can be preset in the software program. Once the hexagons are generated, the center of each hexagon is recommended as a location for placement of a cryoprobe tip, which is the portion of the cryoprobe used for freezing and forming the ice ball, at cryoablation treatment step 210.

The software portion of the computer-assisted planning procedure 200 can also mathematically simulate the freezing process at a cryosurgical simulation step 212 so that the user can “watch” the procedure before performing it. System parameters that will lead to a desired outcome can therefore be confirmed before performing the operation. A guide or template, similar to the type used in brachytherapy, can be used to align cryoprobes with the hexagons at a probe alignment step 214 and guide them into the prostate. Through the use of software including a finite element analysis algorithm, the computer-assisted planning system provides for more accurate cryoprobe placement and more complete cryoablation as the software portion can account for the irregular size and shape of the prostate or other targeted tissue so as to provide for uniform cryoprobe distribution.

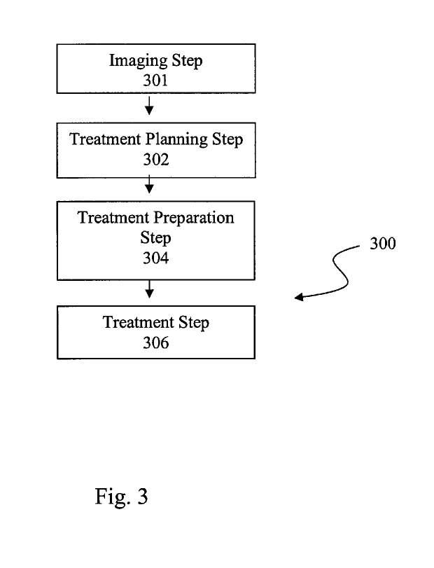

A representative cryosurgical treatment procedure 300 for utilizing cryosurgical system 100 in the cryoablation of the prostate is illustrated in FIG. 3. In performing the cryosurgical treatment procedure 300, a first step generally involves an imaging step 301 in which tumors are identified and located within the prostate. Imaging step 301 can be accomplished with any of a variety of suitable imaging systems including, for example, Magnetic Resonance Imaging (MRI), Computed Tomography Imaging (CT), Near-Infrared Imaging (N-IR), Electrical Impedance Tomography (EIT) and the like, used either individually or in combination.

Once a tumor has been identified and located, a treatment planning step 302 can make use of the computer assisted planning procedure 200 discussed previously can be used to plan and map the prostate. Treatment planning step 302 can include mathematical simulation of the cryosurgical treatment procedure to determine freezing and heating boundary conditions, temperature conditions through the cryoablation process and to recommend locations for insertion of the cryoablation probes.

Following treatment planning step 302, a treatment preparation step 304 can involve prepping the patient and equipment for treatment. Generally, treatment preparation step 304 can include activating the cryosurgical system 100 and positioning the cryosurgical system 100 and related components with respect to the patient. Treatment preparation step 304 can include positioning a needle insertion grid such as, for example, a brachytherapy style grid, with respect to the patient such that insertion of the cryoprobes can be accomplished in accordance with treatment planning step 302.

Once the cryosurgical system 100 is positioned and ready for treatment, a treatment step 306 involving freezing and heating cycles of the inserted cryoprobes is initiated. During the freezing step, iceballs are formed at the tip of the cryoprobes for freezing and consequently killing the targeted tissue of the tumor. During treatment step 306, the size and formation of the iceball must be carefully monitored such that the iceball is freezing only targeted tissue and does not accidentally freeze vital organs or other non-targeted, healthy tissue. Treatment step 306 can further include the use of heating probes to protect certain areas such as nerve bundles or the rectum from freezing.

So as to avoid the previously discussed disadvantages associated with ultrasound imaging, a cryosurgical system according to the present disclosure can also include a non-ultrasound imaging system to track ice ball growth throughout treatment step 306. The non-ultrasound imaging system is advantageous in that shadow regions commonly associated with ultrasound imaging are avoided so as to reduce the potential for damage to healthy tissue or vital organs during treatment step 306.

One representative non-ultrasound imaging system that can be used during treatment step 306 comprises an electrical impedance tomography (EIT) system. With an EIT system, electrodes can be placed on the body or needles positioned within the body. The EIT system then measures the electrical resistance across gaps between the electrodes placed on the body and/or needles placed in the body. Based on the measured electrical resistance, a computer running EIT software can visualize the size and position of the ice ball in real time and without the limitation of shadow regions in proximity to the ice ball.

Another representative imaging system that can be used to monitor iceball growth during treatment step 306 can comprise a near-infrared imaging (N-IR) system. With a N-IR system, light fibers can be placed inside or outside the body and near-infrared absorbance measurements are taken. Based on the absorbance measurements, a computer running N-IR software can be used to visualize the size and position of the ice ball in real time and without the limitation of shadow regions in proximity to the ice ball.

Utilizing either the EIT or N-IR imaging systems, an operator can continually monitor the cryosurgical treatment to ensure that the ice ball is freezing all of the targeted tissue while not contacting the surrounding, healthy tissue during treatment step 306. By using non-ultrasound based imaging systems, physicians other than radiologists can image and perform cryosurgical treatment. Through the use of EIT or N-IR imaging systems including careful positioning of the electrodes and light fibers, a 360 degree view of the ice ball can be generated in real-time as cryosurgical treatment is being performed and the view of the tissue behind the ice ball is not obscured as is commonly encountered with ultrasound based imaging systems.

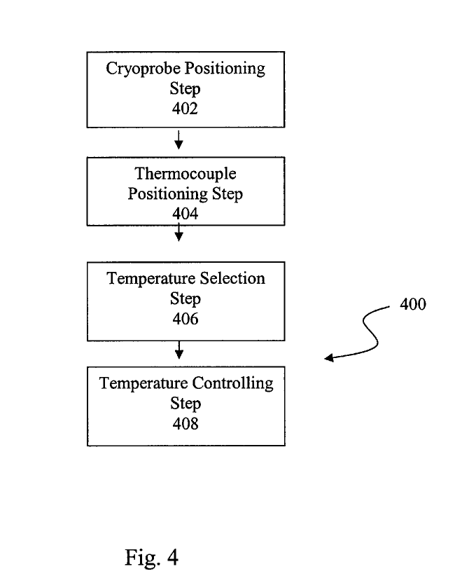

Cryosurgery according to the present disclosure can further be aided through the use of a temperature monitoring system and associated temperature monitoring algorithm 400 that is illustrated in FIG. 4. Through the use of temperature monitoring algorithm 400, temperatures are more evenly controlled within the prostate throughout the cryoablation process and less experience and expertise on the part of the use is necessary to achieve a desired treatment outcome.

Generally, a first step of temperature monitoring algorithm 400 involves a cryoprobe positioning step 402 wherein a plurality of cryoprobes are positioned within identified locations in the prostate. The cryoprobe locations can be identified prior to insertion using the previously discussed computer-assisted planning procedure 200. Preferably, the temperature monitoring system can utilize cryoprobes having servo-actuated valves to selectively control the flow rate of refrigerant gas to the cryoprobes. Next, a thermocouple positioning step 404 involves placing thermocouples into areas where precise temperature control is desired. These areas can include, for example, the urethra, neurovascular bundles, the rectum and the like. Throughout the cryosurgical procedure, the individual thermocouples continually read and transmit temperature data (T_{actual}) to the temperature monitoring system.

Once the cryoprobes and thermocouples are positioned, a user can specify the desired temperature of operation (Tᵤₛₑᵣ) in a temperature selection step 406. Once the user specifies Tᵤₛₑᵣ, a computer running a temperature monitoring software program can begin a temperature controlling step 408 that incorporates the Tᵤₛₑᵣ value as well as the T_{actual} values in a feed back loop that drives the process output proportional to the sum of: 1) a proportionality constant multiplied by the difference between the last read T_{actual} value and the Tᵤₛₑᵣ value (the proportional control); 2) a second proportionality constant multiplied by the difference between the last read T_{actual} value and the integral of the error from the Tᵤₛₑᵣ value (the integral control) and; 3) a third proportionality constant multiplied by the difference between the error between T_{actual} and Tᵤₛₑᵣ at the current time step and at the previous time step (the derivative control). Based on these calculations in the temperature controlling step 408, the temperature monitoring system can continually adjust the servo-actuated valves for each cryoprobe based on the process output after each time step in order to obtain a closer approximation between T_{actual} and Tᵤₛₑᵣ. In some instances, the user may manually adjust the proportionality constants within the temperature monitoring software program in order to obtain a more stable operation.

The above temperature control algorithm 400 gives a higher likelihood of a stable operating temperature. Use of a derivative control alone can yield a process that is sensitive to perturbations or external thermal “noise.” The proportional and integral controls can be used with or without the derivative part of the control. The proportional control is a relatively standard control and the integral control allows correction for bias. The derivative control can allow for even faster response, but may do so at the expense of stability.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A method of performing a cryosurgical procedure, comprising:
imaging a cross-section of target tissue;
tessellating the cross-section with finite element software to define placement positions within the target tissue for one or more cryoprobes;
placing the one or more cryoprobes in the placement positions;
initiating iceball formation at a tip portion of the one or more cryoprobes;
monitoring the iceball formation in real time using a non-ultrasound based imaging method; and
monitoring the temperature at locations within the treated area during the cryosurgical procedure.

2. The method of claim 1, further comprising:
simulating placement of the one or more cryoprobes in the placement positions and initation of iceball formation at the tip portion of one or more cryoprobes prior to placing the one or more cryoprobes in the placement positions.

3. The method of claim 2, further comprising:
modifying one or more parameters of the cryosurgical procedure based on the step of simulating placement of the one or more cryoprobes in the placement positions and initiation of the iceball formation at the tip portion of the one or more cryoprobes..

4. The method of claim 1, further comprising:
aligning a needle grid relative to a patient such that the needle grid corresponds to the placement positions for the one or more cryoprobes.

5. The method of claim 1, wherein the non-ultrasound based imaging method is selected from the group consisting of: electrical impedance tomography and near-infrared imagin.

6. The method of claim 1, further comprising:
identifying a freeze boundary for the target tissue.

7. The method of claim 6, further comprising:
defining an area within the freeze boundary which is not to be treated.

8. The method of claim 1, wherein tessellating the cross-section to define placement positions defines a plurality of hexagons.

9. The method of claim 8, wherein the chord diameter of the hexagons is equal to the size of an iceball that would be generated during iceball formation after a predetermined period of time.

10. The method of claim 9, wherein the predetermined period of time is input by a medial professional.

11. The method of claim 9, wherein the predetermined period of time is automatically preset.

12. A method of controlling temperatures within a prostate during a cryoablation process comprising:
identifying areas in a prostate and surrounding tissue where precise temperature control is desired;
positioning one or more cryoprobes in the prostate wherein an iceball is formed at a tip portion of each cryoprobe;
positioning a thermocouple at each area;
entering a desired operation temperature for each area into a software program;
monitoring actual temperatures for each area with the thermocouples;
comparing the actual temperatures measured by the thermocouples to the desired operation temperature; and
adjusting the temperature of a selected cyroprobe such that the actual temperature approaches the desired operation temperature.

13. The method of claim 12, wherein the step of adjusting the temperature of the selected cryoprobe includes using the desired operation temperature at each location in a feedback loop having an output proportional to a sum comprising a first proportionality constant multiplied by an error measured as the difference between the actual temperature at the thermocouple inserted at the location at a current timestep and the desired operation temperature at the location and a second proportionality constant multiplied by the difference between the actual temperature at the current timestep and the integral of the error.

14. The method of claim 13, wherein the sum further comprises a third proportionality constant multiplied by the difference between the error at the current timestep and the error at a previous timestep.

15. The method of claim 12, wherein the step of adjusting the temperature of the selected cyroprobe includes adjusting a flow rate of refrigerant flowing into the selected cryoprobe.

16. The method of claim 15, wherein the flow rate of refrigerant is adjusted using servo-actuated valves located within the cryoprobes.

17. The method of claim 12, wherein one of the area at which a thermocouple is positioned is selected from the group consisting of: the urethra, a neurovascular bundle, and the rectum.
